(19) United States
(12) Patent Application Publication
(10) Pub. No.: US 2024/0261204 A1
(43) Pub. Date: Aug. 8, 2024

(54) ASCORBIC ACID POLYPEPTIDE DERIVATIVE, PREPARATION METHOD THEREFOR, AND APPLICATION THEREOF

(71) Applicant: HARVEST BIOTECH CO., LTD, Jiaxing, Zhejiang (CN)

(72) Inventors: Junxiang LI, Jiaxing, Zhejiang (CN); Ming XU, Jiaxing, Zhejiang (CN); Yi LU, Jiaxing, Zhejiang (CN); Sining KANG, Jiaxing, Zhejiang (CN)

(21) Appl. No.: 18/557,911

(22) PCT Filed: Apr. 27, 2022
(86) PCT No.: PCT/CN2022/089635

§ 371 Date: Oct. 27, 2023

(30) Foreign Application Priority Data
Apr. 30, 2021 (CN) 202110488537.7

(51) Int. Cl. A61K 8/64; A61Q 19/00; C07K 1/107

(52) U.S. Cl. CPC A61K 8/64; A61Q 19/00; C07K 1/1077

(57) ABSTRACT

Provided in the present disclosure are an ascorbic acid polypeptide derivative, a preparation method therefor, and an application thereof, relating to the technical field of cosmetic raw material development, and the ascorbic acid polypeptide derivative being the compound represented by general formula (1) or a salt thereof. The ascorbic acid polypeptide derivative of a novel structure of the present disclosure has excellent VC whitening and carnosine anti-ageing effects, and solves the stability problem of traditional VC structures. Cell experiments demonstrate that the compound has good biological activity, including reducing ROS, promoting the production of collagen I, reducing tyrosinase activity, and down-regulating melanin secretion. At a concentration of 24.25 μm, the EAC-L-carnosine can down-regulate melanin secretion by 27.85%, greater than the sum of EAC and carnosine at the same concentration. The present invention is a promising anti-ageing active material with ideal anti-ageing effects in terms of skin elasticity, skin tone, and wrinkle reduction.

### CROSS-REFERENCE TO RELATED APPLICATIONS

The present disclosure claims the priority to the Chinese patent application with the filing number “CN 202110488537.7” filed on Apr. 30, 2021 with the Chinese Patent Office, and entitled “Ascorbic Acid Polypeptide Derivative, Preparation Method therefor, and Application thereof”, the contents of which are incorporated herein by reference in entirety.

### TECHNICAL FIELD

The present disclosure relates to the technical field of development of cosmetic raw materials, and in particular to an ascorbic acid polypeptide derivative, a preparation method therefor, and application thereof.

### BACKGROUND ART

With the continuous damage of the earth's ozone layer, ultraviolet rays are increasingly strong, and people are paying more and more attention on methods and raw materials for preventing “photoaging”. UVA is called as invisible killer for skin-aging. Its damage to skin is quite hidden, without significant signs, but is irreversible. UVA not only can cause melanin pigmentation to “blacken” the skin, but also induce ROS generation, to result in loss of collagen, so that the skin gradually ages, and phenomena such as wrinkles, looseness, and black dullness appear. Therefore, it is an efficient anti-aging way to resist both melanin pigmentation and aging of dermal cell level.

L-Ascorbic acid, also known as vitamin C, is a natural water-soluble antioxidant for whitening, and is widely applied to anti-aging ingredient of cosmetics. However, it is sensitive to light and oxygen, and has an unstable structure that limits its application in the cosmetic industry.

In view of this, the present disclosure is specifically proposed.

### SUMMARY OF THE INVENTION

A first objective of the present disclosure is to provide an ascorbic acid polypeptide derivative with a new structure. The ascorbic acid polypeptide derivative of a brand-new structure has excellent efficacies of VC whitening and L-carnosine anti-aging, and solves the stability problem of conventional VC structure.

A second objective of the present disclosure is to provide a preparation method for an ascorbic acid polypeptide derivative.

A third objective of the present disclosure is to provide use of an ascorbic acid polypeptide derivative in the preparation of cosmetics.

A fourth objective of the present disclosure is to provide a cosmetic containing an ascorbic acid polypeptide derivative as an active ingredient.

In order to realize the above objectives of the present disclose, the following technical solutions are particularly adopted.

The present disclosure provides an ascorbic acid polypeptide derivative, being a compound represented by the following general formula (1) or a salt thereof:

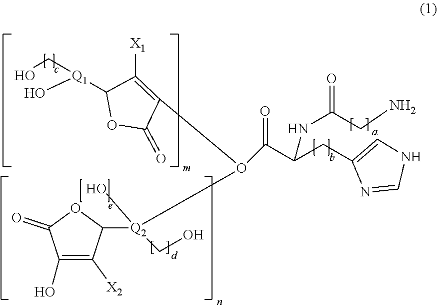

where m is 0 and n is 1, or m is 1 and n is 0;
X₁ and X₂ are independently C1˜C6 alkyl, C1˜C6 alkoxy or halogen;
Q₁ and Q₂ are independently a C1˜C6 alkyl chain;
a, b, c are integers independently selected from 1-10;
e is 0 and d is an integer from 1-10, or, e is 1 and d is 0.

In some embodiments, the ascorbic acid polypeptide derivative is a compound represented by the following general formula (3):

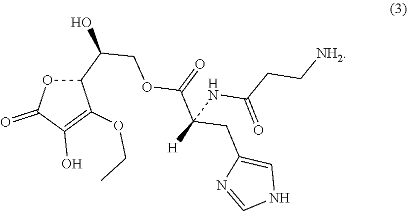

In some embodiments, the ascorbic acid polypeptide derivative is a compound represented by the following general formula (4):

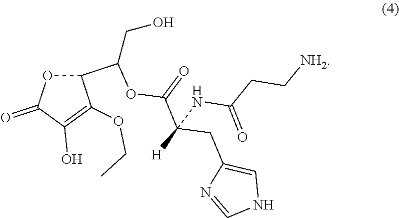

In some embodiments, the ascorbic acid polypeptide derivative is a compound represented by the following general formula (6):

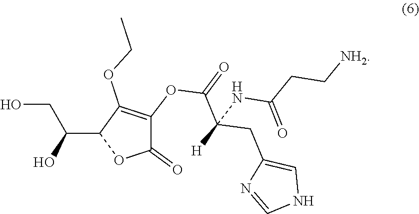

The present disclosure provides a preparation method for the above ascorbic acid polypeptide derivative, including:
first performing carboxyl activation on a raw material A having an amino protecting group, then reacting the same with a raw material B, and removing the protecting group to obtain the ascorbic acid polypeptide derivative, wherein
the raw material A is a compound represented by the following general formula (7):

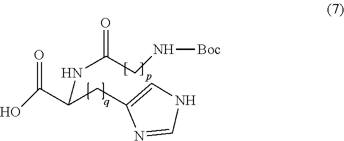

a and b are integers independently selected from 1-10;
the raw material B is a compound represented by the following general formula (8):

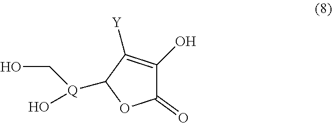

k is an integer selected from 1-10;
Q is a C1˜C6 alkyl chain; and
Y is C1˜C6 alkyl, C1˜C6 alkoxy or halogen.

In some embodiments, the raw material A is L-carnosine having an amino protecting group, and the raw material B is 3-O-ethyl ascorbic acid.

The preparation method includes the following steps:
first reacting the raw material A having an amino protecting group with dicyclohexylcarbodiimide and N-hydroxysuccinimide in an organic solvent, after finishing the reaction, removing by-products, then adding the raw material B and an acid-binding agent to react, and then removing the protecting group, to obtain the ascorbic acid polypeptide derivative.

The present disclosure provides use of the above ascorbic acid polypeptide derivative in the preparation of cosmetics.

The present disclosure provides a cosmetic containing the above ascorbic acid polypeptide derivative as an active ingredient, wherein an effective concentration of the ascorbic acid polypeptide derivative is 10˜50 ppm.

The ascorbic acid polypeptide derivative provided in the present disclosure at least has the following advantageous effects.

In the present disclosure, a structure of ascorbic acid derivative and a polypeptide structure are coupled, and a new ascorbic acid polypeptide derivative is designed and synthesized through rational molecule improvement and modification. The chemical structure is clear and definite.

The ascorbic acid polypeptide derivative of a brand-new structure has a stable structure and high biosafety, and solves the problem that the conventional VC structure is unstable.

After efficacy evaluation, such ascorbic acid derivative demonstrates significant synergistic efficacy in whitening and anti-aging aspects. Cell experiments demonstrate that the compound has good bioactivity, including reducing ROS, promoting production of collagen I, reducing tyrosinase activity, and down-regulating melanin secretion. At a concentration of 24.25 μM, the EAC-L-carnosine can down-regulate melanin secretion by 27.85%, greater than simple superposition of relevant effects of EAC and L-carnosine at the same concentration. The present disclosure is a promising anti-aging active material with ideal anti-aging effects in terms of skin elasticity, skin tone, and wrinkle reduction, and can be widely applied to products such as cosmetics and skin care products.

In addition, the relevant ascorbic acid derivative obtained in the present disclosure also has efficacies such as anti-glycation, anti-blue light, and anti-autophagy, and can be used for skin at various parts (hands, face, limbs, scalp, etc.).

### BRIEF DESCRIPTION OF DRAWINGS

In order to more clearly illustrate the technical solutions in embodiments of the present disclosure or in the prior art, drawings which need to be used in the description of the embodiments or the prior art will be introduced briefly below. Apparently, the drawings in the description below merely show some embodiments of the present disclosure, and those ordinarily skilled in the art still could obtain other drawings in light of these drawings without using any inventive efforts.

FIG. 1 is a diagram of synthesis route of 3-O-ethyl ascorbic acid-L-carnosine in Example 1;

FIG. 2 is an HPLC spectrum of 3-O-ethyl ascorbic acid-L-carnosine synthesized in Example 1;

FIG. 3 is an MS spectrum of 3-O-ethyl ascorbic acid-L-carnosine synthesized in Example 1;

FIG. 4 is a proton nuclear magnetic resonance spectrum of 3-O-ethyl ascorbic acid-L-carnosine synthesized in Example 1;

FIG. 5 is an H,H-COSY spectrum of 3-O-ethyl ascorbic acid-L-carnosine synthesized in Example 1;

FIG. 6 is a result chart of SIRC corneal cell irritation test of Test Example 1;

FIG. 7 is a result chart of corrosive HET-CAM (hen's egg test on chorioallantoic membrane) in Test Example 1;

FIG. 8 is a result chart of enzyme activity test for the tyrosinase in Test Example 2;

FIG. 9 is a result chart of melanin test in Test Example 2, wherein (a) shows test results under different raw materials, and (b) shows test results under different concentrations;

FIG. 10 is a result chart of energy metabolic capability test of keratinocytes in Test Example 2;

FIG. 11 is a result chart of anti-aging repair test for the stratum corneum cell under oxidative stress in Test Example 2;

FIG. 12 is a result chart of immortalized keratinocytes (HaCaT) after UVA-induced damage prevention test in Test Example 2, wherein (a) is a chart of sample cytotoxicity detection; (b) is for cell viability (CSR) after UVA damage prevention; (c) is for expression of intracellular reactive oxygen species (ROS) after UVA damage prevention; and (d) is for expression of intracellular hydroxyproline (HYP) after UVA damage prevention;

FIG. 13 is a result chart of synthesis test of type I collagen of fibroblast of Test Example 2;

FIG. 14 is a result chart of human foreskin fibroblast (HFF) after UVA-induced damage prevention test in Test Example 2, wherein (a) is a chart for cell viability (CSR) after UVA damage prevention; (b) is for expression of intracellular reactive oxygen species (ROS) after UVA damage prevention; and (c) is for expression of intracellular hydroxyproline (HYP) after UVA damage prevention;

FIG. 15 is a result chart of collagen I content of HFF cells under UVA-induced damage;

FIG. 16 is a result chart of skin elasticity values of test-group population in Test Example 3; and

FIG. 17 is a result chart of ITA skin tone of test-group population in Test Example 3.

### DETAILED DESCRIPTION OF EMBODIMENTS

Technical solutions of the present disclosure will be described below clearly and completely in combination with examples. Apparently, the examples described are only some, but not all examples of the present disclosure. Based on the examples in the present disclosure, all of other examples obtained by those ordinarily skilled in the art without using any inventive efforts shall fall within the scope of protection of the present disclosure.

Regulation of melanin generation, collagen synthesis, and cell regeneration activity are important targets in the development of cosmetic active ingredients. The ultra-unstable structure of water-soluble antioxidant L-ascorbic acid (vitamin C) limits its application in the cosmetic industry.

In order to solve this problem, the present disclosure develops an efficient and stable multifunctional ascorbic acid polypeptide derivative. In structure, a highly reduced C═C double bond is retained, and meanwhile, a polypeptide structure is added. Such new ascorbic acid polypeptide not only exhibits stable properties in terms of extracellular matrix enhancement, anti-oxidation, and melanin suppression, etc., but also has significantly improved anti-aging property compared with ascorbic acid and polypeptide.

The present disclosure provides an ascorbic acid polypeptide derivative, being a compound represented by the following general formula (1) or a salt thereof:

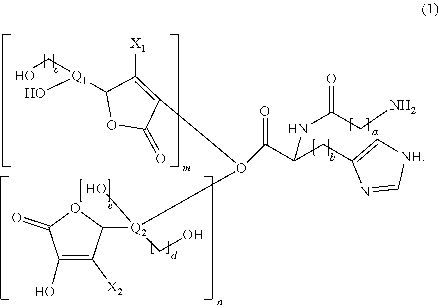

The ascorbic acid polypeptide derivative is a stable derivative formed by binding ascorbic acid and polypeptide organically (via chemical bond).

A compound salt generally refers to a metal salt of compound, such as sodium salt, potassium salt, and the like.
(I) In the above structure of general formula (1): when m is 0 and n is 1, the general formula is:

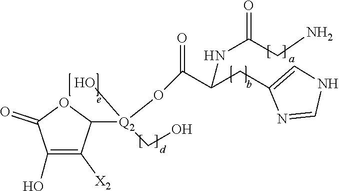

where a is an integer selected from 1-10, a is 1, 2, 3, 4, 5, 6, 7, 8, 9 or 10;
b is an integer selected from 1-10, b is 1, 2, 3, 4, 5, 6, 7, 8, 9 or 10;
e is 0 and d is an integer from 1-10 (d is 1, 2, 3, 4, 5, 6, 7, 8, 9, or 10), or, e is 1 and d is 0;
Q₂ is a C1˜C6 alkyl chain, which may be straight or branched;
X₂ is C1˜C6 alkyl, C1˜C6 alkoxy or halogen;
alkoxy includes, but is not limited to, methoxy, ethoxy, acetoxy, isopropoxy, isopropenyloxy, tert-butoxy, tert-butyl peroxy or butanone oxime;
alkyl includes, but is not limited to, methyl, ethyl, propyl, isopropyl, or butyl (n-butyl, iso-butyl, sec-butyl, tert-butyl); and
halogen includes, but is not limited to, chlorine, bromine, or iodine.

It should be noted that alkyl includes straight or branched alkyl group.

In some typical embodiments, X₂ is ethoxy.

In some typical embodiments, the ascorbic acid polypeptide derivative of (I) is a compound represented by the following general formula (2) or a salt thereof:

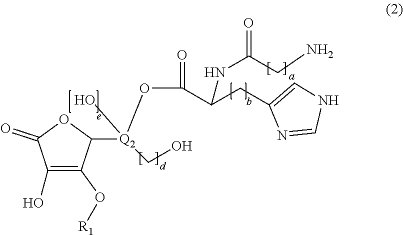

where R₁ is C1˜C6 alkyl, including, but not limited to, methyl, ethyl, propyl, isopropyl, or butyl (n-butyl, iso-butyl, sec-butyl, tert-butyl); and alkyl includes straight or branched alkyl.
a, b are independently integers from 1-5 (a, b are independently 1, 2, 3, 4 or 5); and
e is 0 and d is an integer from 1-5 (d is 1, 2, 3, 4, or 5), or e is 1 and d is 0.

In some typical embodiments, R₁ is ethyl, Q₂ is a C2 alkyl chain, a is 2, b is 1, e is 1 and d is 0, and the ascorbic acid polypeptide derivative is a compound represented by the following general formula (3):

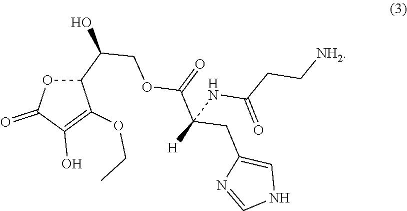

In some other typical embodiments, R₁ is ethyl, Q₂ is a C1 alkyl chain, a is 2, b is 1, e is 0 and d is 1, and the ascorbic acid polypeptide derivative is a compound represented by the following general formula (4):

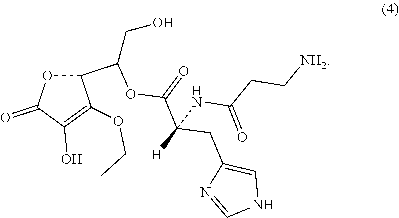

In the above structure of the general formula (1):
(II) when m is 1 and n is 0, the general formula is:

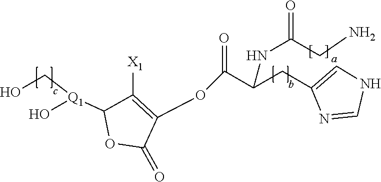

where a is an integer selected from 1-10, a is 1, 2, 3, 4, 5, 6, 7, 8, 9 or 10;
b is an integer selected from 1-10, b is 1, 2, 3, 4, 5, 6, 7, 8, 9 or 10;
c is an integer selected from 1-10, c is 1, 2, 3, 4, 5, 6, 7, 8, 9 or 10;
Q₁ is a C1˜C6 alkyl chain, which may be straight or branched;
X₁ is C1˜C6 alkyl, and C1˜C6 alkoxy or halogen;
alkoxy includes, but is not limited to, methoxy, ethoxy, acetoxy, isopropoxy, isopropenyloxy, tert-butoxy, tert-butyl peroxy or butanone oxime;
alkyl includes, but is not limited to, methyl, ethyl, propyl, isopropyl, or butyl (n-butyl, iso-butyl, sec-butyl, tert-butyl); and alkyl includes straight or branched alkyl group; and
halogen includes, but is not limited to, chlorine, bromine, or iodine.

In some typical embodiments, X₁ is ethoxy.

In some typical embodiments, the ascorbic acid polypeptide derivative of (II) is a compound represented by the following general formula (5) or a salt thereof:

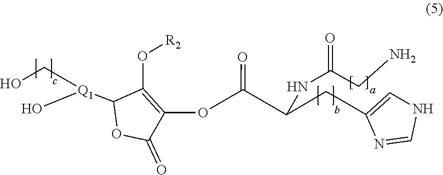

where R₂ is C1˜C6 alkyl, including but not limited to, methyl, ethyl, methylethyl, propyl, isopropyl, or butyl (n-butyl, iso-butyl, sec-butyl, tert-butyl);
Q₁ is a C1˜C6 alkyl chain; and
a, b, c are independently integers from 1-5 (a, b, c are independently 1, 2, 3, 4 or 5).

In some typical embodiments, Q₁ is C1 alkyl chain, R₂ is ethyl, a is 2, b, c are independently 1, and the ascorbic acid polypeptide derivative is a compound represented by the following general formula (6):

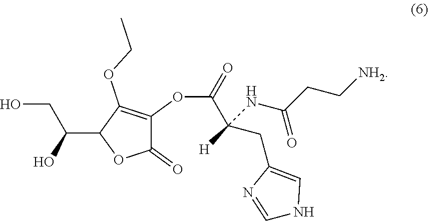

The present disclosure further provides a preparation method for the above ascorbic acid polypeptide derivative, including:
first performing carboxyl activation on a raw material A having an amino protecting group, then reacting the same with a raw material B, and removing the protecting group to obtain the ascorbic acid polypeptide derivative, wherein
the raw material A is a compound represented by the following general formula (7):

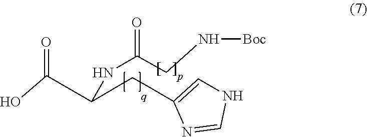

p is an integer selected from 1-10, p is 1, 2, 3, 4, 5, 6, 7, 8, 9 or 10;
q is an integer selected from 1-10, q is 1, 2, 3, 4, 5, 6, 7, 8, 9 or 10;
the raw material B is a compound represented by the following general formula (8):

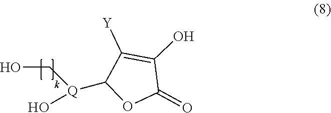

k is an integer selected from 1-10, k is 1, 2, 3, 4, 5, 6, 7, 8, 9 or 10;
Q is a C1˜C6 alkyl chain; and
Y is C1˜C6 alkyl, C1˜C6 alkoxy or halogen.

Alkoxy includes, but is not limited to, methoxy, ethoxy, acetoxy, isopropoxy, isopropenyloxy, tert-butoxy, tert-butyl peroxy or butanone oxime.

Alkyl includes, but is not limited to, methyl, ethyl, methylethyl, propyl, isopropyl, or butyl (n-butyl, iso-butyl, sec-butyl, tert-butyl), and the alkyl includes straight or branched alkyl group.

Halogen includes, but is not limited to, chlorine, bromine, or iodine.

In some typical embodiments, the raw material A is L-Carnosine having an amino protecting group;

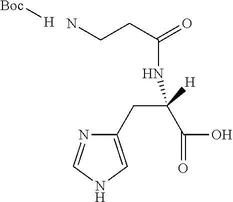

the raw material B is 3-O-ethyl ascorbic acid (EAC);

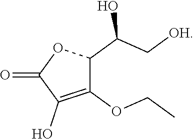

In some typical embodiments, the compounds represented by the general formulas (3), (4), and (6), or a salt thereof can be prepared according to the following reaction:

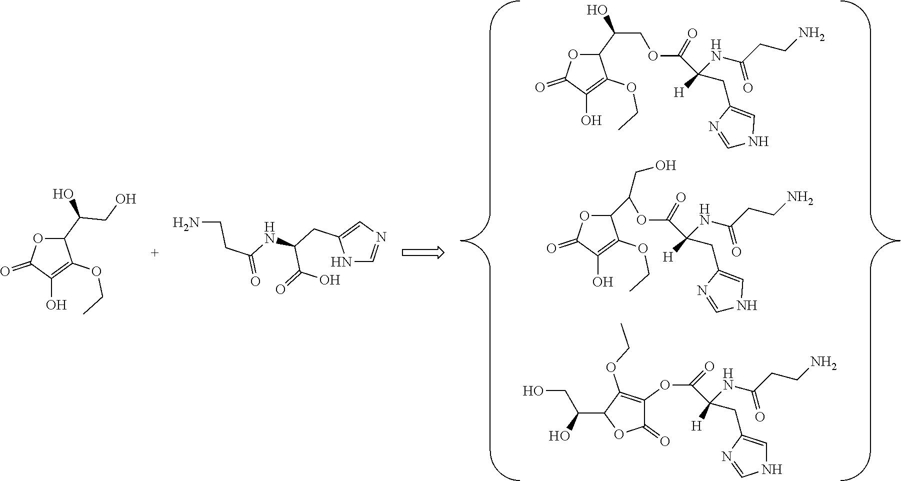

The preparation method includes the following steps:
first reacting carnosine having an amino protecting group with dicyclohexylcarbodiimide and N-hydroxysuccinimide in an organic solvent, after finishing the reaction, removing by-products, then adding 3-O-ethyl ascorbic acid and an acid-binding agent to react, and then removing the protecting group, to obtain the ascorbic acid polypeptide derivative.

Carboxyl of the L-carnosine is activated with dicyclohexylcarbodiimide (DCC) and N-hydroxysuccinimide (NHS).

The organic solvent includes, but is not limited to, DMF, 1,4-dioxane, and acetone.

The acid-binding agent includes, but is not limited to, N,N-diisopropylethylamine, diisopropylethylamine (EDIPA), and triethylamine.

Reaction temperature and reaction time may generally be 1-30 h, optionally 12-18 h, and the reaction temperature may generally be 5-50° C., optionally 15-35° C.

The preparation method is as follows:
(a) dissolving Boc-L-Caroline in DMF, adding DCC and NHS, reacting at room temperature for 12-18 h, and filtering to remove solid by-products (N,N-dicyclohexylurea); and
(b) to filtrate adding 3-O-ethyl ascorbic acid ether and DIPEA, reacting at room temperature for 1-5 h, concentrating, drying, and recrystallizing, to obtain a compound with a protecting group represented by the general formulas (3), (4), and (6), and removing the protecting group to obtain a product EAC-L-Carnosine.

The present disclosure further provides use of the above ascorbic acid polypeptide derivative in the preparation of cosmetics.

The present disclosure further provides a cosmetic containing the above ascorbic acid polypeptide derivative as an active ingredient, wherein an effective concentration of the ascorbic acid polypeptide derivative is 10˜50 ppm.

The compound of the present disclosure is finally developed by effectively binding the structure of the ascorbic acid derivative and the polypeptide structure (typically, effective ingredients vitamin VC (L-ascorbic acid) and L-carnosine (β-alanyl-L-histidine), and performing rational molecule improvement, modification, and design. The ascorbic acid polypeptide derivative of a brand-new structure has excellent efficacies of VC whitening and L-carnosine anti-aging, and solves the stability problem of conventional VC structure.

Safety and efficacy performance of the innovative polypeptide derivative are evaluated through various cells, semi-in vivo models, and population efficacy evaluation, which proves that good whitening and anti-aging synergistic efficacies can be obtained at a relatively low effective concentration (24.25 μM/10 ppm), including stimulating collagen synthesis by L-carnosine and L-ascorbic acid, inhibiting ROS and melanin generation, etc. A cosmetic sample is prepared with a scientific formula, a rational volunteer efficacy test is carried out, and the anti-aging activity of this compound is verified. It is proved (10˜50 ppm) that the ascorbic acid polypeptide derivative of a brand-new structure can be applied to use scene of cosmetics under the condition of meeting economic conditions.

The present disclosure is further described below with examples. Unless otherwise specified, materials in the examples were prepared according to existing methods, or purchased directly from the market.

Reagents used: DCC, C₁₃H₂₂N₂, dicyclohexylcarbodiimide, dehydrating agent; NHS, N-Hydroxysuccinimide, also known as HOSu, C₄H₅NO₃; DMF, dimethylformamide, hydroscopicity, miscible with water and most organic solvents; DIPEA, N,N-diisopropylethylamine, basic organic solvent; 1,4-dioxane, solvent, stabilizer.

### Example 1 Synthesis of 3-O-Ethyl Ascorbic Acid-L-Carnosine

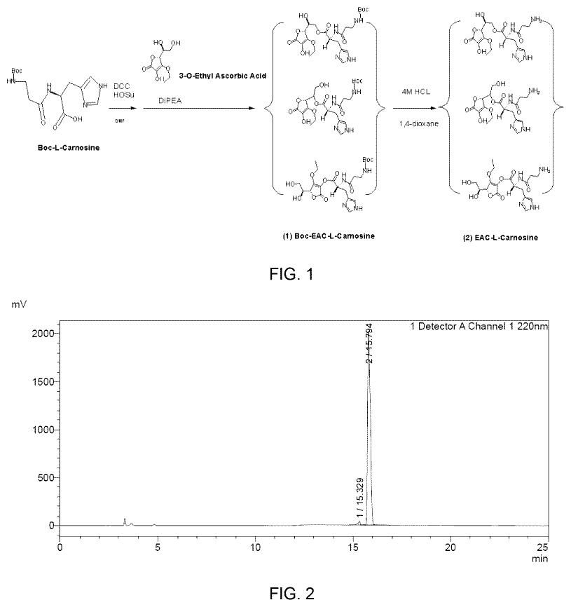

Synthesis route of 3-O-ethyl ascorbic acid-L-carnosine is as shown in FIG. 1, including the following steps:
1. firstly, dissolving Boc-L-Carnosine (13 g, 39.9 mmoL) in DMF (400 mL);
2. secondly, adding DCC (7.98 g, 38.8 mmol) and HOSu (4.45 g, 38.8 mmol);
3. after that, stirring solution at room temperature for about 15 h, and filtering to remove solid by-products (N,N-dicyclohexylurea);
4. next, to filtrate adding 3-O-ethyl ascorbic acid ether (8.2 g, 40.2 mmol) and DIPEA (16 mL, 92 mmol);
5. subsequently, stirring mixture at room temperature for 3 h, followed by concentration, and vacuum-drying;
6. recrystallizing solid with methanol and isopropyl ether to obtain Boc-EAC-L-Carnosine (16.4 g, 32 mmol) as a light yellow solid;
7. dissolving Boc-EAC-L-Camosine in 1,4-dioxane (100 mL), and adding 4M hydrochloric acid and 1,4-dioxane (99 mL, 396 mmol);
8. stirring mixture at room temperature for 4 h, then concentrating, grinding and purifying the solid with acetone and isopropanol to obtain EAC-L-Carnosine (3-O-ethyl ascorbic acid-L-carnosine) (5.2 g, 98%); and
9. after lyophilization, the EAC-L-Carnoline being a white powder (yield: 4.9%).

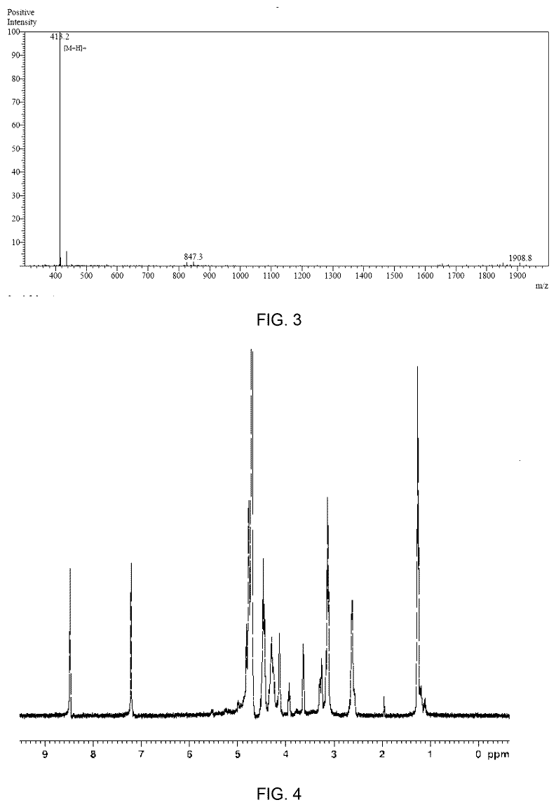

HPLC spectrum of 3-O-ethyl ascorbic acid-L-carnosine is as shown in FIG. 2. Detection conditions: pump A: 0.065% trifluoroacetic acid in 100% water (v/v); pump B: 0.05% trifluoroacetic acid in 100% acetonitrile (v/v); total flow: 1 ml/min; and wavelength: 220 nm.

### LC Time Program:

| Time | Module | Command | Value |
|---|---|---|---|
| 0.01 | Pump | B. Conc | 0 |
| 5.00 | Pump | B. Conc | 0 |
| 20.00 | Pump | B. Conc | 30 |
| 25.00 | Pump | B. Conc | 65 |
| 25.01 | Pump | B. Conc | 95 |
| 27.00 | Pump | B. Conc | 95 |
| 27.01 | Pump | B. Conc | 0 |
| 35.00 | Pump | B. Conc | 0 |
| 35.01 | Controller | Stop | — |

Chromatography column: Inertsil ODS-3 4.6×250 mm; and
Detection device: SS-CM-0309.

Peaking is as shown in the table below:

### Detector a Channel 1 220 nm

| Peak Number | Peak time | Area | Height | Area ratio % |
|---|---|---|---|---|
| 1 | 15.329 | 346699 | 40972 | 1.729 |
| 2 | 15.794 | 19711028 | 2016121 | 98.271 |
| Total | | 20057727 | 2057092 | 100.000 |

MS spectrum of 3-O-ethyl ascorbic acid-L-carnosine is as shown in FIG. 3. Detection conditions: interface: ESI; atomization flow: 1.5 L/min; CDL temperature: 250; block temperature: 200; device: GK11010007; interface bias: +4.5 kV; dry gas flow: 5 L/min; flow: 0.2 ml/min; B. conc: 50% H₂O/50% MeOH.

Proton nuclear magnetic resonance spectrum of 3-O-ethyl ascorbic acid-L-carnosine is as shown in FIG. 4 (400 MHZ, solvent D₂O).

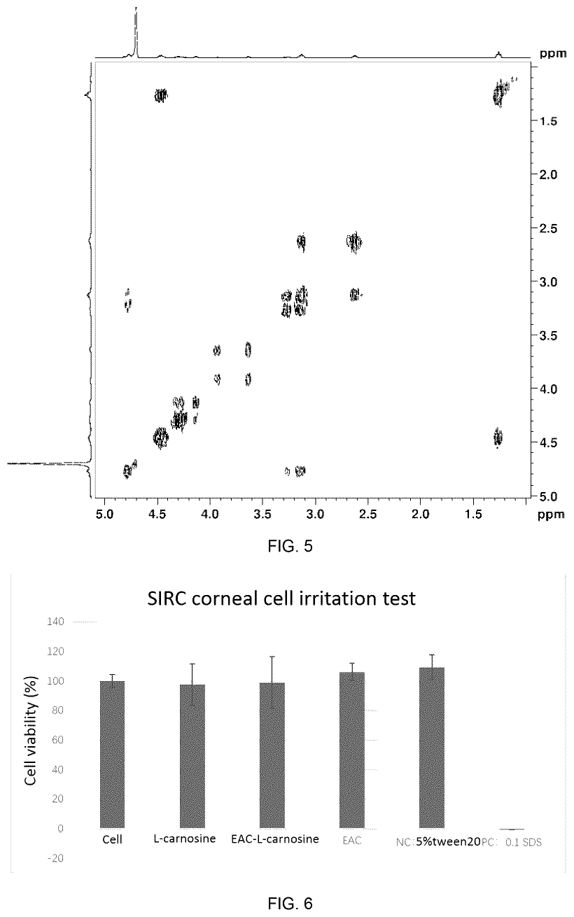

H,H-COSY spectrum of 3-O-ethyl ascorbic acid-L-carnosine is as shown in FIG. 5 (400 MHZ, solvent D₂O).

Substance synthesized in Example 1 was used as a cosmetic raw material, and safety and efficacy thereof were evaluated with a plurality of cell models, including biosafety evaluation of corneal cells in vitro (SIRC), ROS content reduction test evaluated by antioxidant activity of human keratinocyte (HaCaT), collagen synthesis evaluation by skin fibroblasts (FB) in vivo as well as tyrosinase activity and melanin regulation evaluation of melanin to melanoma cell (B16) model. In vivo studies of anti-aging effect verification were also conducted using VISIA CR and CK instrument.

### Test Example 1 Safety Evaluation Test

Substance synthesized in Example 1 completed the following safety animal alternative test under the company's own cell model platform, as shown in the following table:

| | | Test | |
|---|---|---|---|
| | Test Item/System | Conclusion | Execution Standard |
| Cytological | Acute toxicity NHK test | pass | OECD 129 |
| Model Safety | Ocular irritation SIRC model | pass | OECD 491 |
| Test | Ocular irritation HET-CAM model | pass | SN329-2009 |
| | Phototoxicity 3T3 cell model | pass | GB/T 21769-2008 |
| | Blood cell hemolysis test model | pass | Toxicology in Vitro 13 |
| | | | (1999) 343~354 |
| Human Body | Skin patch test and trial test | pass | GB17149.2-1997 |
| Safety Test | | | |

### (1) SIRC Corneal Cell Irritation Test:

Raw material synthesized in Example 1 was formulated into a 10 ppm solution sample, and detected according to industrial standard SN/T 3084.2-2014 of the Entry-exit Inspection and Quarantine of People's Republic of China. Result was CSR=106.0, indicating that the sample had no irritant.

Detection method was rabbit corneal cell SIRC cell viability test. To-be-tested cells were washed 1˜2 times with PBS. 100 μL of MEM complete medium and 10 μL of CCK-8 solution were added to each well, and placed in a carbon dioxide incubator, for incubation of 2˜4 h. The absorbancy (OD) value was measured at 450 nm wavelength. CCK-8 reagent can be replaced with MTT.

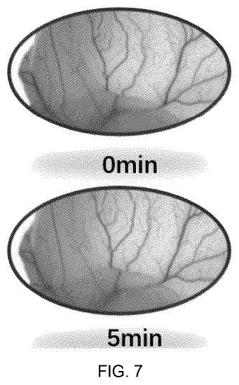

Cell viability of different samples is as shown in FIG. 6.

### (2) Ocular Irritant/Corrosive HET-CAM of Cosmetics:

The raw material synthesized in Example 1 was formulated into a 10 ppm solution sample, and detected according to industrial standard SN/T 2329-2009 of the Entry-exit Inspection and Quarantine of People's Republic of China. Result was IS=0, indicating a negative reaction, and the sample being non-irritant.

Result of the corrosive HET-CAM is as shown in FIG. 7.

It was verified that aqueous solution of the raw material had no adverse reaction at the lowest effective concentration, and had very good biosafety.

### Test Example 2 Efficacy Evaluation Test

### (1) Whitening Efficacy

A method for in vitro testing whitening efficacy for B16 mouse melanoma cells was designed according to Association Standard T/SHRH 021-2019 for cosmetics whitening efficacy test, in combination with NCBI and CNKI literature research:

### 1) Enzyme Activity Test for Tyrosinase:

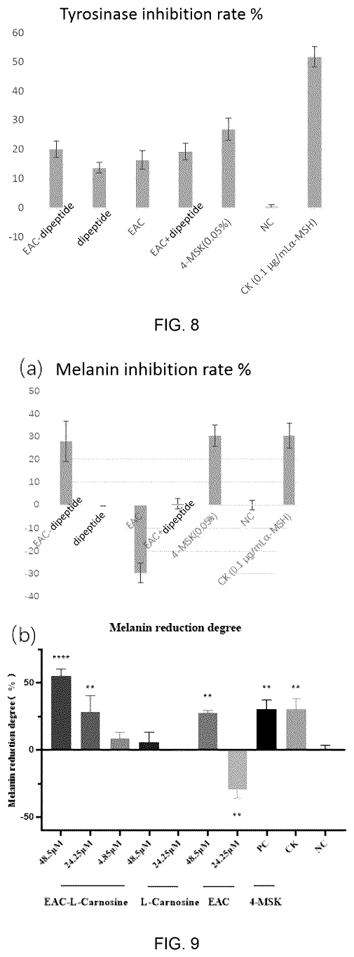

Cell samples having been treated by an inducer (α-MSH) and a to-be-tested substance were placed in a 96-well cell culture plate, for repeated freeze-thawing lysis. To each well 100 μL of 0.1˜0.5% levodopa was added, incubation was performed in a 37° C. cell incubator for about 2 h. the absorbancy (OD) value was measured at 450 nm wavelength. Results are as shown in FIG. 8.

### 2) Measurement of Melanin Content:

Cell samples having been treated by the inducer (α-MSH) and the to-be-tested substance were placed in a 6-well cell culture plate. 0.5˜2 mL of cell lysate was added. The culture plate was allowed to stand in a 65° C. constant-temperature oven for 2 h. Then, after the lysate was uniformly mixed, the lysate was added to a 96-well assay plate at 150 μL/well. The absorbancy (OD) value was measured at 450 nm wavelength. Results are as shown in FIG. 9.

The test proved that this raw material can effectively inhibit melanin generation and tyrosinase enzyme activity. At the same concentration (24.25 μM/10 ppm), EAC-L-carnosine has better capability of inhibiting tyrosinase than classical VC derivative monomer 3-O-ethyl ascorbic acid ether (EAC for short, a more stable ascorbic acid derivative), dipeptide monomer (L-carnosine), and physical mixture of the two (EAC+L-carnosine), and has significantly improved capability of inhibiting final melanin. The inhibitory effect is close to 0.05% of Shiseido's patented ingredient 4-MSK (model inducer concentration: 0.1 μg/mL (63 nM) of α-MSH).

It can be seen that EAC-L-carnosine has significant whitening efficacy, and is superior to gold standard ingredients such as 3-O-ethyl ascorbic acid and potassium 4-methoxysalicylate (4-MSK) in some indexes.

EAC-L-Carnosine has a significant effect of inhibiting melanin secretion at 24.25 μM (about 10 ppm). At 24.25 UM concentration, the effect of EAC-L-Carnosine is the best. It is speculated that it may be related to the reducibility of EAC-L-Carnosine, L-carnosine structure, and cell proliferation.

### (2) Anti-Oxidation and Anti-Aging

### 1) Energy Metabolic Capability Test of Keratinocytes:

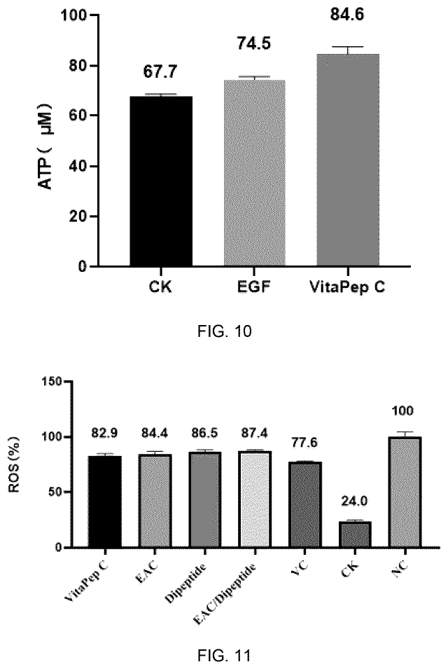

It was tested whether ATP in HaCaT cells would be affected by EAC-L-carnosine. Human immortalized keratinocytes HaCaT having been treated with the test substance were fully lysed using a certain amount of cell lysate, and centrifuged at 4° C. and 12000 g for 5 min. Supernatant was taken, and cell lysis samples of each experimental group were added to a 96-well assay plate (opaque) at 20 μL/well. ATP detection solution was further added at 100 μL/well. The plate was left to stand at room temperature for 3˜5 min. Self-luminescence intensity was detected using a full-waveband plate reader. Results are as shown in FIG. 10.

The results prove that compared with blank group, 24.25 μM EAC-L-carnosine could improve ATP expression by 25.0%, indicating that it has the effect of significantly up-regulating ATP, i.e. the effect of improving cell activity. (Note: in an experimental positive control group 1 nM EGF used in the present study, and the raw material is a drug, at a test concentration of 1 nM, and cannot be used in a skin care product). The raw material of the present disclosure can significantly enhance the energy metabolic capability of keratinocytes.

### 2) Anti-Aging Repair Test for Stratum Corneum Cell Under Oxidative Stress

In order to study the anti-aging repair efficacy of the raw material of the present disclosure to stratum corneum cell under oxidative stress, according to Anti-wrinkly and Firming Lifting Efficacy Test of Cosmetics—In vitro Test Method of Reactive Oxygen Species (ROS) Inhibition with keratinocytes (Association Standard T/SHRH 032-2020), oxidative damage cell models were constructed with H₂O₂-induced HaCaT cells, and relative proliferation rate and ROS expression quantity of the cells were respectively measured.

After human immortalized keratinocytes HaCat were plated, when the degree of cell fusion was around 65%, original culture medium was removed, and H₂O₂ culture medium at a certain concentration was used, and they were placed in a carbon dioxide incubator for 2 h. After induction was completed, the original culture medium was removed, and replaced with a culture medium containing to-be-tested sample (or a positive sample), and they were placed in a carbon dioxide incubator.

ROS detection: after 24 h, a certain amount of DCFH-DA working solution was used in replacement. Incubation was performed in the dark for 15˜30 min. Washing was performed for 3˜5 times with a serum-free culture medium. 100 μL of serum-free culture medium was added. Fluorescence intensity after stimulation was detected at 488 nm excitation wavelength and 525 emission wavelength.

Cell viability detection: after 24 h, a serum-free culture medium containing 10% of CCK-8 was used in replacement. After incubation of 2˜4 h in a carbon dioxide incubator, an OD value was detected at 450 nm wavelength.

Results are as shown in FIG. 11, showing ROS expression in immortalized keratinocytes (HaCat) after 24 h of sample repair after hydrogen peroxide (H₂O₂) damage.

Experimental results indicate that the raw material can effectively repair oxidative damage, significantly improve cell activity, and significantly down-regulate the ROS expression quantity. At the same concentration, compared with VC derivative (EAC), L-carnosine, and synergistic effect of physical mixture of L-carnosine and EAC, the overall efficacy is more excellent, and the effect of “1+1>2” is embodied.

### 3) Anti-Aging Repair Test for Stratum Corneum Cell Under UVA Photoaging Stress:

After human immortalized keratinocytes HaCat were plated, when the degree of cell fusion was around 65%, original culture medium was removed, and the original culture medium was replaced with PBS containing the to-be-tested sample. The plate was placed under a specific ultraviolet device, and UVA irradiation dose was 15 J/cm². After the induction was completed, the drug-containing PBS was removed, and replaced with a culture medium containing the to-be-tested sample, and they were placed in a carbon dioxide incubator.

ROS detection: after 24 h, a certain amount of DCFH-DA working solution was used in replacement. Incubation was performed in the dark for 15˜30 min. Washing was performed for 3˜5 times with serum-free culture medium. 100 μL of serum-free culture medium was added. Fluorescence intensity after stimulation was detected at 488 nm excitation wavelength and 525 emission wavelength.

Cell viability detection: after 24 h, a serum-free culture medium containing 10% of CCK-8 was used in replacement. After incubation of 2˜4 h in a carbon dioxide incubator, an OD value was detected at 450 nm wavelength.

HaCat with damage induced by a certain dose of UVA was chosen, to construct a keratinocyte UVA damage cell model.

FIG. 12 is a plot of experimental data of UVA damage to HaCaT cell, and conclusion is as follows.

The EAC-L-carnosine (about 24 μM/10 ppm) of the present disclosure, without cytotoxicity, has the effects of up-regulating the activity of keratinocytes after UVA irradiation, significantly inhibiting the generation of free radical ROS, and significantly activating collagen precursor hydroxyproline HYP. The above index levels are all superior to the effect of VC monomer (24 μM), and close to the positive control TGF-β1 (100 ng/ml).

### 4) Synthesis Test of Type I Collagen of Fibroblast

According to Anti-wrinkly and Firming Lifting Efficacy Test of Cosmetics—In vitro Test Method of Collagen I Contents with fibroblasts (Association Standard T/SHRH 032-2020), human primary fibroblasts FB were used to measure influence of each raw material group on collagen secretion of FB at a concentration (24.25 μM/10 ppm).

After human primary fibroblasts FB were plated, when the degree of cell fusion was 45%˜65%, original culture medium was removed, the original culture medium was replaced with PBS containing the to-be-tested sample. The plate was placed in a carbon dioxide incubator for incubation for 24 h+2 h. After incubation was finished, 200 μL of cell culture supernatant was collected per well in a 1.5 mL sterile centrifuge tube, and stored frozen in an ultra-low temperature freezer at −80° C.

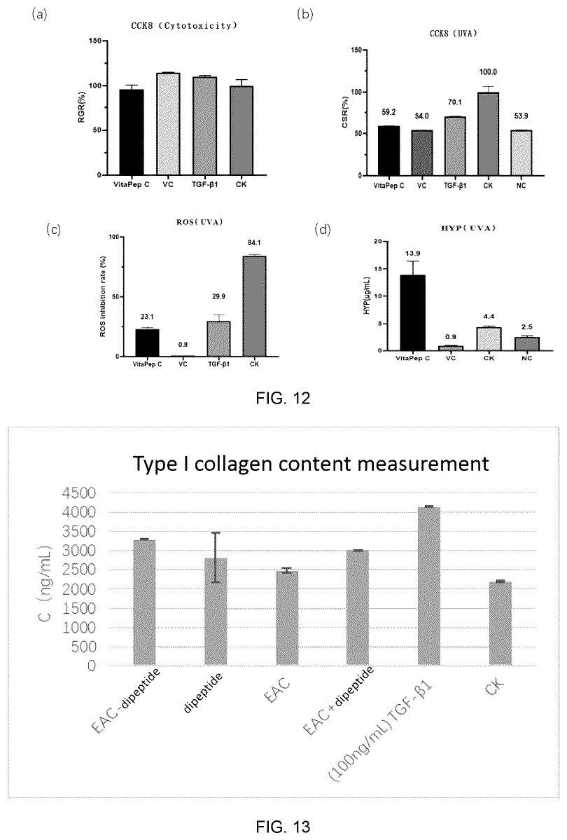

Human type I collagenase immunization kit was used for ELISA detection. First, a standard was used for detection and a standard curve was plotted, and then a collagen content contained in the fibroblast supernatant diluted at an appropriate ratio was measured. Results are as shown in FIG. 13.

The results prove that, at the same concentration, the EAC-L-carnosine has a more significant effect on promoting collagen generation compared with classical EAC, dipeptide monomer, and mixture of the two. In the above, TGF-B1 was a positive control at a concentration of 100 ng/ml. It can be seen that EAC-L-carnosine can significantly enhance the synthesis of fibroblast type I collagen.

### 5) Fibroblast Anti-Aging Repair Test Under UVA Photoaging Stress

After HFF human foreskin fibroblast were plated, when the degree of cell fusion was 65˜85%%, original culture medium was removed, and the original culture medium was replaced with PBS containing the to-be-tested sample. The plate was placed under a specific ultraviolet device, and UVA irradiation dose was 5 J/cm². After the induction, the drug-containing PBS was removed, replaced with a culture medium containing the to-be-tested sample, and they were placed in a carbon dioxide incubator.

ROS detection: after 24 h, a certain amount of DCFH-DA working solution was used in replacement. Incubation was performed in the dark for 15˜30 min. Washing was performed for 3˜5 times with serum-free culture medium. 100 μL of serum-free culture medium was added. Fluorescence intensity after stimulation was detected at 488 nm excitation wavelength and 525 emission wavelength.

Cell viability detection: after 24 h, a serum-free culture medium containing 10% of CCK-8 was used in replacement. After incubation of 2˜4 h in a carbon dioxide incubator, an OD value was detected at 450 nm wavelength.

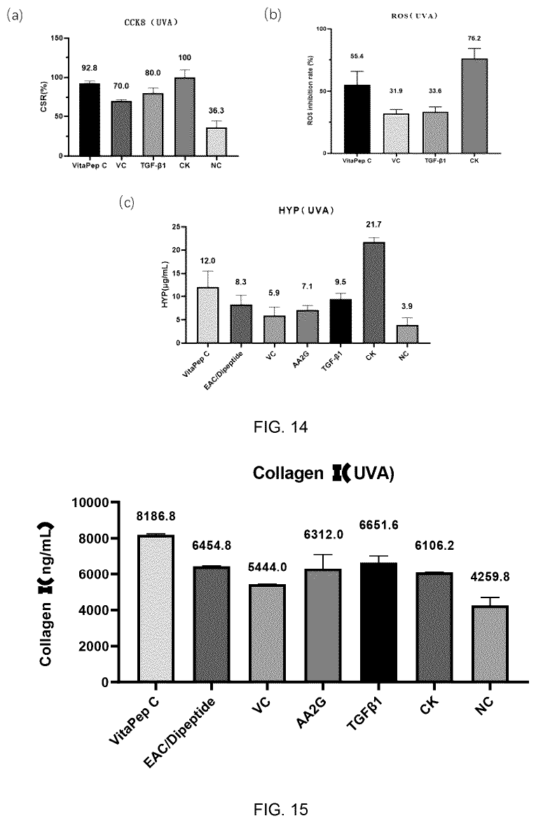

HYP (cell lysate) content detection: a hydroxyproline (HYP) content detection kit was used. First, a HYP standard was used for detection and a standard curve was plotted, and then a HYP (cell supernatant) content contained in the fibroblast lysate diluted at an appropriate ratio was measured. Results are as shown in FIG. 14.

Type I collagen content detection: human type I collagenase (Col I) immunization kit was used for ELISA detection. First, a type I collagen standard was used for detection and a standard curve was plotted, and then a collagen content contained in the fibroblast supernatant diluted at an appropriate ratio was measured.

Experimental results of the type I collagen content of HFF cells with UVA-induced damage treated by Graphpad Prism 8.0.2 software are shown in FIG. 15.

The EAC-L-carnosine (about 24 μM/10 ppm), without cytotoxicity, has the effects of significantly up-regulating the activity of fibroblasts after UVA irradiation, significantly inhibiting the generation of free radical ROS, and significantly activating collagen precursor hydroxyproline HYP. The above index levels are all superior to the synergistic effect of the mixed addition of VC monomer/EAC monomer and L-carnosine, and the effect of the patented VC derivative AA2G, and is close to the positive control TGF-β1.

It can be seen that the EAC-L-carnosine has a significant anti-aging repair effect on multiple dimension indexes, is superior to simple superposition of the effect of physical mixture of classical L-carnosine and VC derivative, and has a synergistic anti-aging effect, thereby achieving the effect of “1+1>2”.

### Test Example 3 Efficacy for Actual Volunteer Population

Test method: adding 20 ppm EAC-dipeptide raw material to a basic cream, using the same twice per week, and testing the effect in 4 weeks and 8 weeks, wherein a basic cream without EAC-dipeptide raw material was taken as a blank placebo.

Test population: experimental group of 15 people, placebo group of 15 people (31-54 years old, female).

Method of use: volunteers used the cream once every morning and evening, with 1 g per use, and uniformly applied the cream on the whole face.

Test instrument: CK instrument.

Test method: using cutometer and Colorimeter probes of the CK instrument to respectively perform a skin elasticity test and a skin tone test on a random side cheek of each volunteer, in a constant-temperature and constant-humidity environment, at 0 weeks, and 4 weeks and 8 weeks after use of the product.

Ingredients of the blank placebo and test cream are shown in tables below.

| Blank Placebo | | | Test Cream | | |
|---|---|---|---|---|---|
| | | Proportion | | | Proportion |
| | INCI | (%) | | INCI | (%) |
| 1 | Water | 66.10 | 1 | Water | 66.10 |
| 2 | — | 0.00 | 2 | EAC-dipeptide | 20 ppm |
| 3 | Stearic acid | 15.00 | 3 | Stearic acid | 15.00 |
| 4 | Ethylhexyl | 2.00 | 4 | Ethylhexyl | 2.00 |
| | palmitate | | | palmitate | |
| 5 | Lanolin | 1.00 | 5 | Lanolin | 1.00 |
| 6 | Cetearyl alcohol | 0.50 | 6 | Cetearyl alcohol | 0.50 |
| | Cetearyl | | | Cetearyl | |
| | glucoside | | | glucoside | |
| 7 | C14-22 alcohol | 0.60 | 7 | C14-22 alcohol | 0.60 |
| | C12-20 alkyl | | | C12-20 alkyl | |
| | glucoside | | | glucoside | |
| 8 | Butanediol | 10.00 | 8 | Butanediol | 10.00 |
| 9 | Sorbitol | 3.00 | 9 | Sorbitol | 3.00 |
| 10 | Potassium | 1.00 | 10 | Potassium | 1.00 |
| | hydroxide | | | hydroxide | |
| 11 | Phenoxyethanol | 0.50 | 11 | Phenoxyethanol | 0.50 |
| 12 | Methylparaben | 0.20 | 12 | Methylparaben | 0.20 |
| 13 | (Daily use) flavor | 0.10 | 13 | (Daily use) flavor | 0.10 |
| | | 100.00 | | | 100.00 |

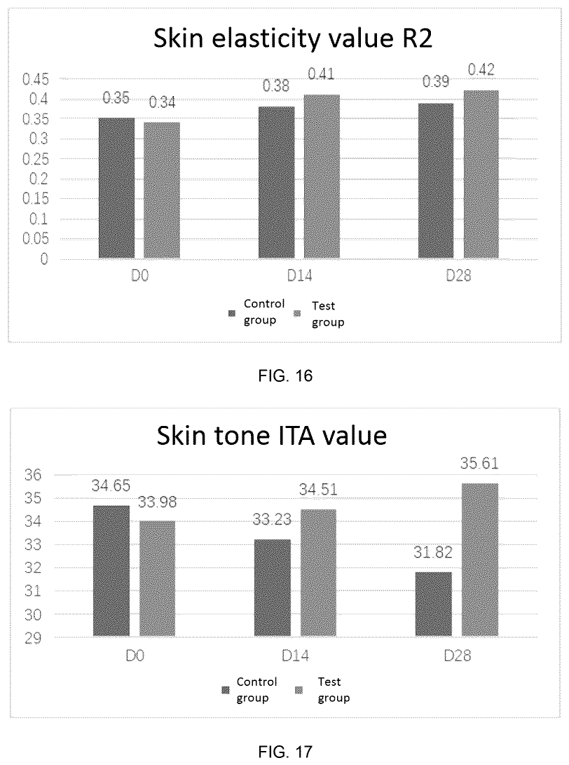

Results of skin elasticity values of the test group population are shown in FIG. 16. The results show that the skin elasticity value R₂ of the test group population had a significant trend of elevation on the 14th and 28th days. An elevation magnitude of the placebo group was lower than that of the test group, proving the significant anti-aging efficacy of the EAC-dipeptide.

Results of ITA skin tone of the test group population are shown in FIG. 17. The results show that the ITA skin tone of the test group population had a significant trend of elevation on the 14th and 28th days. The tone of the placebo control group was slightly decreased, proving the effect of whitening and brightening skin color of the EAC-dipeptide.

Conclusion: cell experiment proves that the compound 3-O-ethyl ascorbic acid-L-carnosine has good bioactivity, including reducing ROS, promoting generation of collagen I, reducing activity of tyrosinase, and down-regulating secretion of melanin. Specifically, at a concentration of 24.25 UM, the EAC-L-carnosine can down-regulate the secretion of melanin by 27.85%, which is greater than a sum of EAC and L-carnosine at the same concentration, proving significant synergistic efficacies in whitening and anti-aging dimensions. In some critical indexes, the derivative exhibits the efficacy that is superior to VC and L-carnosine monomer as well as their physical compounding. In addition, the research results of human volunteers support that 3-O-ethyl ascorbic acid-L-carnosine has an ideal anti-aging effect in terms of skin elasticity, skin tone, and wrinkle reduction, and has multiple functions (anti-aging, whitening, anti-oxidation, anti-inflammation, etc.), thus it is a promising anti-aging active substance.

Finally, it should be explained that the various examples above are merely used for illustrating the technical solutions of the present disclosure, rather than limiting the present disclosure; although the detailed description is made to the present disclosure with reference to various preceding examples, those ordinarily skilled in the art should understand that they still could modify the technical solutions disclosed in various preceding examples, or make equivalent substitutions to some or all of the technical features therein; and these modifications or substitutions do not make the corresponding technical solutions essentially depart from the scope of the technical solutions of various examples of the present disclosure.

1. An ascorbic acid polypeptide derivative, wherein the ascorbic acid polypeptide derivative is a compound represented by a following general formula (1) or a salt thereof: 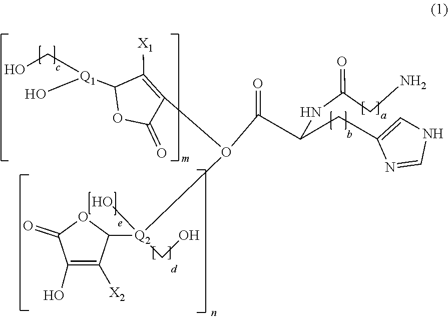
where m is 0 and n is 1, or m is 1 and n is 0;
X₁ and X₂ are independently C1˜C6 alkyl, C1˜C6 alkoxy or halogen;
Q₁ and Q₂ are independently a C1˜C6 alkyl chain;
a, b, c are integers independently selected from 1-10; and
e is 0 and d is an integer from 1-10, or, e is 1 and d is 0.

2. The ascorbic acid polypeptide derivative according to claim 1, wherein the ascorbic acid polypeptide derivative is a compound represented by a following general formula (2) or a salt thereof: 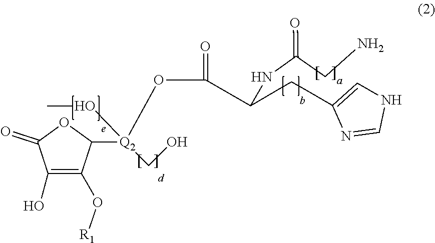
where R₁ is C1˜C6 alkyl;
Q₂ is a C1˜C6 alkyl chain;
a, b are independently integers from 1-5; and
e is 0 and d is an integer from 1-5, or e is 1 and d is 0.

3. The ascorbic acid polypeptide derivative according to claim 2, wherein the ascorbic acid polypeptide derivative is a compound represented by a following general formula (3):

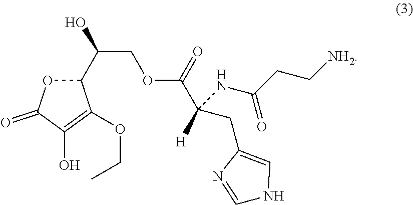

4. The ascorbic acid polypeptide derivative according to claim 2, wherein the ascorbic acid polypeptide derivative is a compound represented by a following general formula (4):

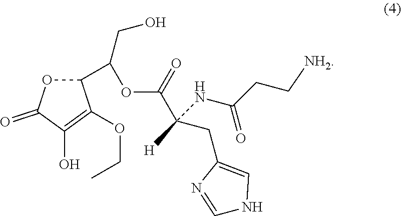

5. The ascorbic acid polypeptide derivative according to claim 1, wherein the ascorbic acid polypeptide derivative is a compound represented by a following general formula (5) or a salt thereof: 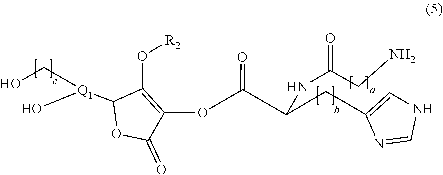
where R₂ is C1˜C6 alkyl;
Q₁ is a C1˜C6 alkyl chain; and
a, b, c are independently integers from 1-5.

6. The ascorbic acid polypeptide derivative according to claim 5, wherein the ascorbic acid polypeptide derivative is a compound represented by a following general formula (6):

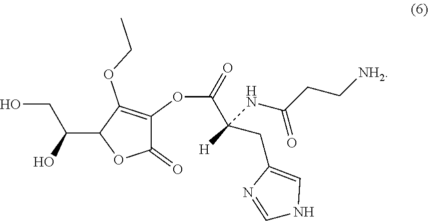

7. A preparation method for the ascorbic acid polypeptide derivative according to claim 1, comprising:
first performing carboxyl activation on a raw material A having an amino protecting group, then reacting the raw material A with a raw material B, and removing the protecting group to obtain the ascorbic acid polypeptide derivative, wherein
the raw material A is a compound represented by a following general formula (7): 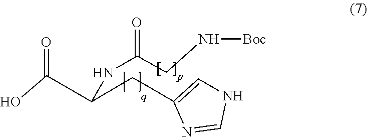
p, q are integers independently selected from 1-10;
the raw material B is a compound represented by a following general formula (8): 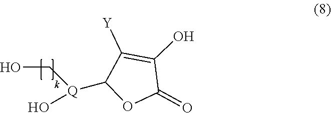
k is an integer selected from 1-10;
Q is a C1˜C6 alkyl chain; and
Y is C1˜C6 alkyl, C1˜C6 alkoxy or halogen.

8. The preparation method according to claim 7, wherein the raw material A is L-carnosine having the amino protecting group; and the raw material B is 3-O-ethyl ascorbic acid;
the preparation method comprises following steps:
first reacting the raw material A having the amino protecting group with dicyclohexylcarbodiimide and N-hydroxysuccinimide in an organic solvent, after finishing the reaction, removing by-products, then adding the raw material B and an acid-binding agent to react, and then removing the protecting group, to obtain the ascorbic acid polypeptide derivative.

9. (canceled)

10. A cosmetic, containing the ascorbic acid polypeptide derivative according to claim 1 as an active ingredient, wherein an effective concentration of the ascorbic acid polypeptide derivative is 10˜50 ppm.
